(19) United States
(12) Patent Application Publication
(10) Pub. No.: US 2013/0267147 A1
(43) Pub. Date: Oct. 10, 2013

(54) BREAST CUSHION AND PRODUCTION METHOD THEREOF

(71) Applicant: Shuangyin Xia, Shanghai (CN)

(72) Inventor: Shuangyin Xia, Shanghai (CN)

(73) Assignee: SHANGHAI FOLIAGE INDUSTRY CO., LTD., Shanghai (CN)

(21) Appl. No.: 13/991,624

(22) PCT Filed: Sep. 2, 2011
(86) PCT No.: PCT/CN2011/079297

§ 371 Date: Jun. 5, 2013

(30) Foreign Application Priority Data
Dec. 29, 2010 (CN) 201010611928.5

(51) Int. Cl. A61F 13/14; A41C 5/00

(52) U.S. Cl. CPC A61F 13/141; A41C 5/00
USPC 450/37; 450/92

(57) ABSTRACT

A bra cup shaped breast cushion comprises a first impermeable membrane layer (1), a hygroscopic material layer (2), a nonwoven fabric layer (3) and a second impermeable membrane layer (4). The first impermeable membrane layer (1) adheres to the nonwoven fabric layer (3) to form a cavity. At least one set of elastic components (5) are placed at the adhering part between the first impermeable membrane layer (1) and the nonwoven fabric layer (3). The hygroscopic material layer (2) is located in the cavity. There is at least one notch (6) on the edge of the hygroscopic material layer (2). The second impermeable membrane layer (4) adheres to the nonwoven fabric layer (3) to form a pocket shape. The hygroscopic material layer (2) with the notch (6) is more easily formed into the shape of a bra cup under the tension force of the elastic components (5), so as to make the breast cushion fit the breast. The elastic components (5) have a certain elasticity, so they can adapt to users with different breast shapes to some extent. When breast milk spillage is relatively large and cannot be absorbed quickly by the hygroscopic material layer (2), part of the breast milk can be stored in the pocket formed by the second impermeable membrane layer (4) and the nonwoven fabric layer (3).

The present application claims the benefit of priority to Chinese Patent Application No. 201010611928.5 titled “BREAST CUSHION AND PRODUCTION METHOD THEREOF”, filed with the Chinese State Intellectual Property Office on Dec. 29, 2010, the entire disclosure of which is herein incorporated by reference.

### FIELD OF THE INVENTION

The present application relates to a breast cushion and a method for producing the breast cushion.

### BACKGROUND OF THE INVENTION

A breast cushion is a kind of product which is put in a bra for absorbing breast milk leaked so as to prevent clothes from being stained.

A conventional breast cushion is generally a circular paper or cotton sheet, and when the breast cushion is put into a cup of a bra, multiple wrinkles will be appeared, so that the breast cushion is not apt to fit the breast. Hence, a cup-shaped breast cushion is in demand.

### SUMMARY OF THE INVENTION

In view of this, an object of the present application is to provide a breast cushion and a method for producing the breast cushion.

In order to achieve the above object, the present application provides technical solutions as follows.

A breast cushion is in a cup shape in whole, and includes a first impermeable membrane layer, a wadding paper layer, a hygroscopic material layer, a non-woven fabric layer, and a second impermeable membrane layer, wherein:

the first impermeable membrane layer adheres to the non-woven fabric layer so as to form a cavity, at least one set of elastic components are further provided between the adhering parts of the first impermeable membrane layer and of the non-woven fabric layer;

the hygroscopic material layer is located in the cavity, at least one notch is provided on the edge of the hygroscopic material layer; and

the second impermeable membrane layer adheres to the non-woven fabric layer so as to form a pocket.

A method for producing a breast cushion includes:

processing a hygroscopic material to be in a sheet shape;

making at least one notch on the edge of the sheet-like hygroscopic material so as to form a hygroscopic material layer;

covering the upper and lower surfaces of the hygroscopic material layer with a non-woven fabric and a first impermeable membrane respectively, with the peripheries of the non-woven fabric and of the first impermeable membrane extending outwards beyond the hygroscopic material layer;

placing at least one set of elastic components between the parts of the non-woven fabric and of the first impermeable membrane beyond the hygroscopic material layer;

adhering the parts of the non-woven fabric and of the first impermeable membrane beyond the hygroscopic material layer together; and

adhering the peripheries of the second impermeable membrane layer and of the non-woven fabric layer together so as to form a pocket.

It may be seen from the above technical solutions that, the hygroscopic material layer having the notch is easier to he deformed and formed into a cup shape under the tension force of the elastic components so as to enable the breast cushion to fit the breast. Also, since the elastic components have a certain elasticity, they can be adapted to users with different breast shapes to some extent. Furthermore, when breast milk leakage is relatively large and cannot be absorbed quickly by the hygroscopic material layer, a part of the breast milk can be stored in the pocket formed by the second impermeable membrane layer and the non-woven fabric layer.

### BRIEF DESCRIPTION OF THE DRAWINGS

In order to illustrate embodiments of the present application or the technical solutions in the prior art more clearly, drawings required in the description of the embodiments and the prior art will be described briefly hereinafter. Apparently, the drawings described hereinafter are only some embodiments of the present application, and other drawings may also be acquired according to these drawings by those skilled in the art without a creative work.

FIG. 1 is a schematic structural view of a breast cushion according to an embodiment of the present application;

FIG. 2 is a side view of FIG. 1;

FIG. 3 is a schematic structural view of a hygroscopic material layer according to an embodiment of the present application;

FIG. 4 is another schematic structural view of a breast cushion according to an embodiment of the present application;

FIG. 5 is yet another schematic structural view of a breast cushion according to an embodiment of the present application;

FIG. 6 is still another schematic structural view of a breast cushion according to an embodiment of the present application;

FIG. 7 is further another schematic structural view of a breast cushion according to an embodiment of the present application; and

FIG. 8 is a flow chart of a method for producing a breast cushion according to an embodiment of the present application.

### DETAILED DESCRIPTION OF THE INVENTION

The technical solutions in the embodiments of the present application will be described clearly and completely hereinafter in conjunction with the accompanying drawings in the embodiments of the present application. Obviously, the embodiments described are only part of the embodiments of the present application and not all. Based on the embodiments in the present application, all other embodiments acquired by those skilled in the art without making any creative efforts all fall into the protection scope of the present application.

A breast cushion is a kind of product which is put in a bra fir absorbing breast milk leaked so as to prevent clothes from being stained.

A conventional breast cushion is generally a circular paper or cotton sheet, and when the breast cushion is put into a cup of a bra, multiple wrinkles will be appeared, so that the breast cushion is not apt to fit the breast. Hence, a cup-shaped breast cushion is in demand.

In view of this, an embodiment of the present application provides a cup-shaped breast cushion.

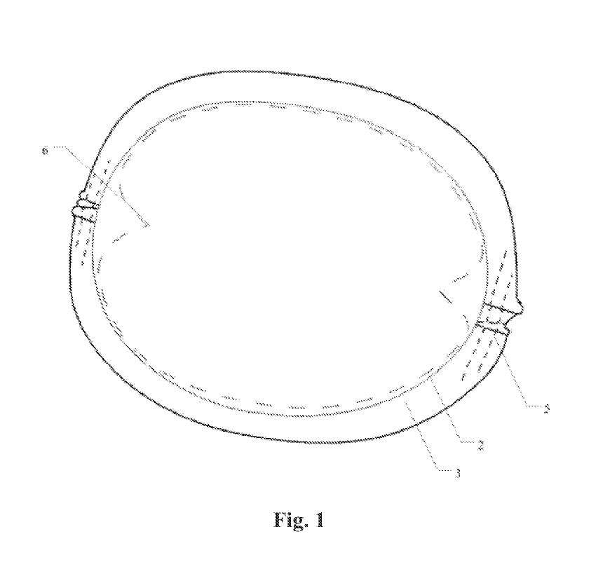

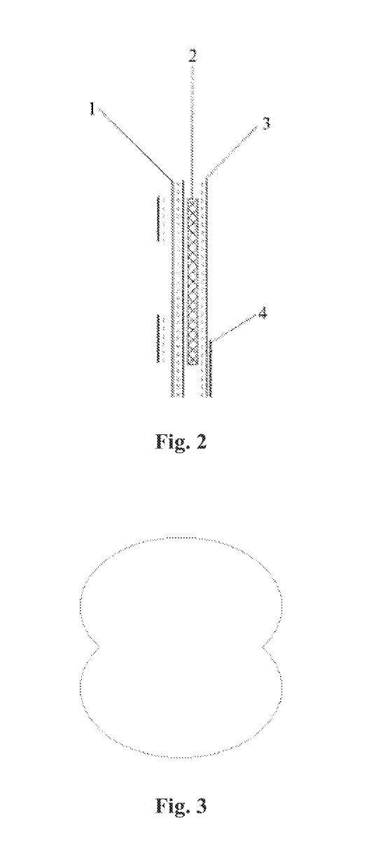

FIGS. 1 and 2 show a structure of the above breast cushion including a first impermeable membrane layer 1, a hygroscopic material layer 2 (indicated by the dotted line in FIG. 1), a non-woven fabric layer 3, and a second impermeable membrane layer 4, wherein:

the first impermeable membrane layer 1 adheres to the non-woven fabric layer 3 to form a cavity, at least one set of elastic components 5 are further provided between the adhering parts of the first impermeable membrane layer 1 and the non-woven fabric layer 3;

the hygroscopic material layer 3 is located in the cavity, at least one notch 6 is provided on the edge of the hygroscopic material layer 3, for example, in the present embodiment, there are two notches 6 each being in a V shape, referring to FIG. 3, so that the entire hygroscopic material layer 3 is like “8”; and

the second impermeable membrane layer 4 adheres to the non-woven fabric layer 3 so as to have a pocket shape.

The material adopted in the hygroscopic material layer is fluff pulp and SAP (Super Absorbent Polymer).

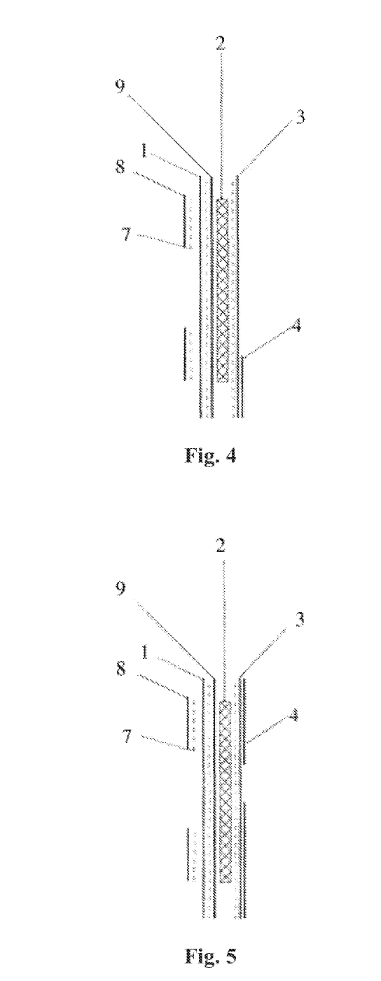

In another embodiment of the present application, referring to FIG. 4, the above breast cushion may further include a double-faced adhesive tape 7 provided on the first impermeable membrane layer 1. Thus, when being used, the release paper 8 on the double-faced adhesive tape 7 may be torn down, and the breast cushion may be adhered in the bra.

In another embodiment of the present application, also please referring to FIG. 4, a wadding paper layer 9 may further be provided in the above cavity.

It may be seen from the above technical solutions, the hygroscopic material layer 3 having the notch 6 is easier to be deformed and formed into a cup shape under the tension force of the elastic components 5 so as to enable the breast cushion to fit the breast. Also, since the elastic components 5 have a certain elasticity, they can be adapted to users with different breast shapes to some extent. Furthermore, when breast milk leakage is relatively large and cannot be absorbed quickly by the hygroscopic material layer 3, part of the breast milk can be stored in the pocket formed by the second impermeable membrane layer 4 and the non-woven fabric layer 3.

The function of the above second impermeable membrane layer 4 is substantially to form a pocket together with the non-woven fabric layer 3. In an embodiment of the present application, the above second impermeable membrane layer 4 may be half-moon shaped in whole. When the breast cushion is used, the placement of the breast cushion needs to be adjusted, so as to enable the second impermeable membrane layer 4 to be located under the nipple, thus accumulating the redundant milk.

Of course, in another embodiment of the present application, referring to FIG. 5, the overall shape of the second impermeable membrane layer 4 may also match the non-woven fabric layer 3, and a hole through which the nipple passes is provided in the second impermeable membrane layer 4. Thus, the nipple may contact the non-woven fabric layer 3 through the hole and, when the milk is relatively large, the milk may still flow into the pocket formed by the second impermeable membrane layer 4 and the non-woven fabric layer 3.

Now, the relationship between the notch 6 and the elastic components 5 is emphatically introduced.

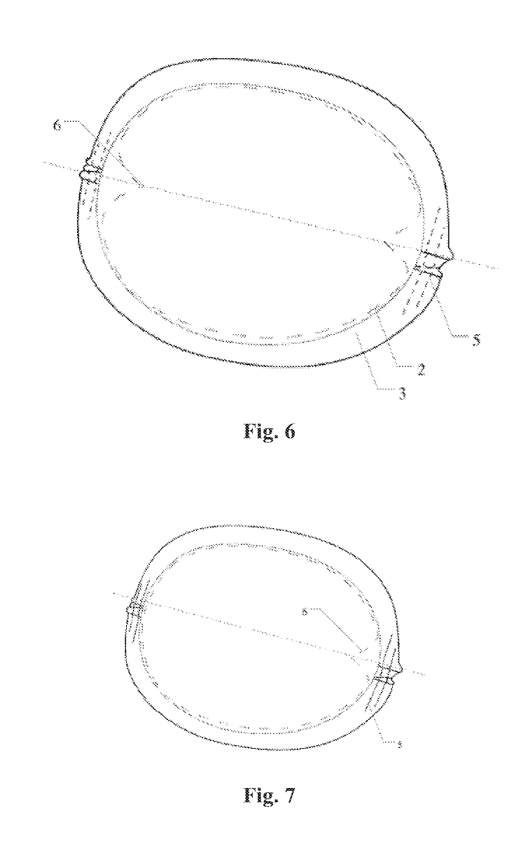

Referring to FIG. 6, in an embodiment of the present application, there may be two notches 6, and two sets of the elastic components 5 each including two elastic components 5. The central connecting line of the two notches 6 is coincident with the central connecting line of the two sets of elastic components 5.

Referring to FIG. 7, in another embodiment of the present application, there are one notch 6, and two sets of the elastic components 5, and the central connecting line of the two sets of elastic components 5 passes through the center of the notch.

Alternatively, in another embodiment of the present application, there are two elastic components 5, and one set of the notches 6, and the connecting line between the centers of the elastic components 5 and the centers of the notches 6 passes through the center of the hygroscopic material layer 3.

Alternatively, in another embodiment of the present application, there are one notch 6, and one set of the elastic components 5, and the connecting line between the center of the notch 6 and the centers of the elastic components 5 passes through the center of the hygroscopic material layer 3.

Of course, in practice, it is not likely to be exactly coincident or pass the centers.

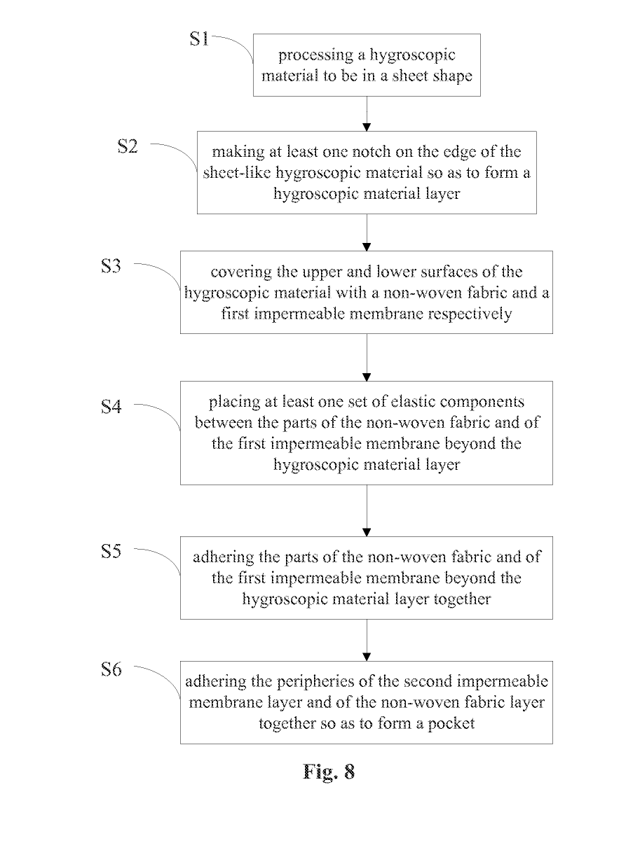

Correspondingly, a method for producing the breast cushion is also disclosed in an embodiment of the present application. Referring to FIG. 8, the method at least includes:

S1, processing a hygroscopic material to be in a sheet shape;

S2, making at least one notch on the edge of the sheet-like hygroscopic material so as to form a hygroscopic material layer;

S3, covering the upper and lower surfaces of the hygroscopic material with a non-woven fabric (i.e. the above non-woven fabric layer) and a first impermeable membrane (i.e. the above first impermeable membrane layer) respectively, with the peripheries of the non-woven fabric and of the first impermeable membrane extending outwards beyond the hygroscopic material layer;

S4, placing at least one set of elastic components between the parts of the non-woven fabric and of the first impermeable membrane beyond the hygroscopic material layer;

S5, adhering the parts of the non-woven fabric and of the first impermeable membrane beyond the hygroscopic material layer together; and

S6, adhering the peripheries of the second impermeable membrane layer and of the non-woven fabric layer together so as to form a pocket.

In other embodiments of the present application, the above step S5 may be specifically implemented by hot pressing and bonding the parts of the non-woven fabric and of the first impermeable membrane beyond the hygroscopic material layer.

In other embodiments of the present application, the above step S6 may be specifically implemented by hot pressing and bonding the peripheries of the second impermeable membrane layer and of the non-woven fabric layer.

The embodiments are described herein in a progressive manner. Description of each of the embodiments is mainly focused on differences between the embodiment and the other embodiments, and the same or similar elements between the embodiments may be cross-referenced.

The above description of the disclosed embodiments enables those skilled in the art to implement or use the present disclosure. Various modifications to those embodiments will be apparent for those skilled in the art. The general principle defined herein may be implemented in other embodiments without departing from the spirit or the scope of the present disclosure. Accordingly, the present disclosure will not be limited to those embodiments illustrated herein, but will fall into the widest scope which conforms to the principle and novel features disclosed herein.

1. A breast cushion, having a cup shape in whole, and comprising a first impermeable membrane layer, a hygroscopic material layer, a non-woven fabric layer, and a second impermeable membrane layer, wherein:
the first impermeable membrane layer adheres to the non-woven fabric layer so as to form a cavity, at least one set of elastic components are further provided between adhering parts of the first impermeable membrane layer and of the non-woven fabric layer;
the hygroscopic material layer is located in the cavity, at least one notch is provided on the edge of the hygroscopic material layer; and
the second impermeable membrane layer adheres to the non-woven fabric layer so as to form a pocket.

2. The breast cushion according to claim 1, further comprising a double-faced adhesive tape provided on the first impermeable membrane layer.

3. The breast cushion according to claim 2, wherein there are provided two notches and two sets of the elastic components, and a connecting line between centers of the two notches is coincident with a connecting line between centers of the two sets of elastic components.

4. The breast cushion according to claim 2, wherein there are provided one notch and two sets of the elastic components, and a connecting line between centers of the two sets of elastic components passes through a center of the notch.

5. The breast cushion according to claim 2, wherein there are provided one notch and one set of the elastic components, and a connecting line between a center of the notch and centers of the elastic components passes through a center of the hygroscopic material layer.

6. The breast cushion according to claim 1, wherein the second impermeable membrane layer is half-moon shaped in whole.

7. The breast cushion according to claim 1, wherein an overall shape of the second impermeable membrane layer matches the non-woven fabric layer, and a hole through which a nipple passes is provided in the second impermeable membrane layer.

8. A method for producing a breast cushion, comprising:
processing a hygroscopic material to be in a sheet shape;
making at least one notch on the edge of the sheet-like hygroscopic material so as to form a hygroscopic material layer;
covering an upper surface and a lower surface of the hygroscopic material layer with a non-woven fabric and a first impermeable membrane respectively, with peripheries of the non-woven fabric and of the first impermeable membrane extending outwards beyond the hygroscopic material layer;
placing at least one set of elastic components between parts of the non-woven fabric and of the first impermeable membrane beyond the hygroscopic material layer;
adhering the parts of the non-woven fabric and of the first impermeable membrane beyond the hygroscopic material layer together; and
adhering peripheries of a second impermeable membrane layer and of the non-woven fabric layer together so as to form a pocket.

9. The method according to claim 8, wherein the step of adhering the parts of the non-woven fabric and of the first impermeable membrane beyond the hygroscopic material layer together is implemented by hot pressing and bonding the parts of the non-woven fabric and of the first impermeable membrane beyond the hygroscopic material layer.

10. The method according to claim 8, wherein the step of adhering the peripheries of the second impermeable membrane layer and of the non-woven fabric layer together is implemented by hot pressing and bonding the peripheries of the second impermeable membrane layer and of the non-woven fabric layer.

11. The breast cushion according to claim 2, wherein the second impermeable membrane layer is half-moon shaped in whole.

12. The breast cushion according to claim 3, wherein the second impermeable membrane layer is half-moon shaped in whole.

13. The breast cushion according to claim 4, wherein the second impermeable membrane layer is half-moon shaped in whole.

14. The breast cushion according to claim 5, wherein the second impermeable membrane layer is half-moon shaped in whole.

15. The breast cushion according to claim 2, wherein an overall shape of the second impermeable membrane layer matches the non-woven fabric layer, and a hole through which a nipple passes is provided in the second impermeable membrane layer.

16. The breast cushion according to claim 3, wherein an overall shape of the second impermeable membrane layer matches the non-woven fabric layer, and a hole through which a nipple passes is provided in the second impermeable membrane layer.

17. The breast cushion according to claim 4, wherein an overall shape of the second impermeable membrane layer matches the non-woven fabric layer, and a hole through which a nipple passes is provided in the second impermeable membrane layer.

18. The breast cushion according to claim 5, wherein an overall shape of the second impermeable membrane layer matches the non-woven fabric layer, and a hole through which a nipple passes is provided in the second impermeable membrane layer.
